(19) United States
(12) Patent Application Publication
(10) Pub. No.: US 2015/0232899 A1
(43) Pub. Date: Aug. 20, 2015

(54) Multi-Enzymatic Preparation Containing the Secretome of an Aspergillus Japonicus Strain

(71) Applicants: INSTITUT NATIONAL DE LA RECHERCHE AGRONOMIQUE, Paris (FR); IFP ENERGIES NOUVELLES, Rueil Malmaison (FR); CENTRE NATIONAL DE LA RECHERCHE SCIENTIFIQUE, Paris (FR); UNIVERSITE D'AIX MARSEILLE, Marseille Cedex 07 (FR)

(72) Inventors: Jean-Guy Berrin, Roquevaire (FR); David Navarro, Marseille (FR); Nicolas Lopes-Ferreira, Croisilles (FR); Antoine Margeot, Paris (FR); Pedro Coutinho, Marseille (FR); Bernard Henrissat, Marseille (FR)

(21) Appl. No.: 14/427,228

(22) PCT Filed: Sep. 10, 2013
(86) PCT No.: PCT/IB2013/058435

§ 371 Date: Mar. 10, 2015

(30) Foreign Application Priority Data
Sep. 10, 2012 (FR) 1258457

(51) Int. Cl. C12P 19/02; C12P 7/10; C12N 9/42

(52) U.S. Cl. CPC C12P 19/02; C12N 9/2437; C12N 9/2445; C12P 7/10

(57) ABSTRACT

The invention relates to a multi-enzymatic preparation containing the secretome of the CNCM I-4639 strain of Aspergillus japonicus. This secretome, which contains in particular cellulases and hemicellulases, can be used for the saccharification of lignocellulosic substrates, in particular in combination with the secretome of Trichoderma reesei.

The present invention relates to improving the saccharification of lignocellulosic biomass.

Lignocellulose is a major constituent of plant biomass, and is the subject of major interest as a starting material for the production of various chemical products, especially fermentable simple sugars resulting from the hydrolysis (generally known as saccharification) of its polysaccharide constituents. At the present time, the main product of saccharification of lignocellulosic biomass is glucose, which may be converted by ethanolic fermentation into ethanol, which may be used as biofuel.

Lignocellulose consists mainly of three types of polymer, in variable proportions depending on the plant species: cellulose, hemicellulose and lignin. These constituents are linked together via various types of bonds, covalent and non-covalent.

Cellulose represents up to 45% of the dry weight of lignocellulose. It is composed of linear chains of D-glucose units linked together via β-1,4-glucoside bonds, these chains being linked together via hydrogen bonds or de van der Waals forces.

Hemicelluloses are heteropolymers representing 15% to 35% of plant biomass, and containing pentoses (β-D-xylose, α-L-arabinose), hexoses (β-D-mannose, β-D-glucose, α-D-galactose) and uronic acids.

Lignin is a complex heteropolymer, consisting of phenylpropane units linked together via various types of bonds. Lignin is linked both to hemicellulose and to cellulose, coating them in a complex three-dimensional structure which makes them sparingly accessible to hydrolysis.

To date, the route considered as being the most promising for the saccharification of lignocellulose is enzymatic hydrolysis, using enzymes produced by cellulolytic microorganisms, especially filamentous fungi. This hydrolysis is preceded by a pretreatment of the biomass, the aim of which is to reduce the complexity of the lignocellulosic network, especially by dissolving the lignin and/or hemicellulose, reducing the crystallinity of cellulose or increasing its area accessible to hydrolysis. This pretreatment may be performed by various techniques, such as mechanical milling, thermolysis, treatment with a dilute acid, with a base or with a peroxide, steam explosion, etc. (for a review, see Hendriks A. T., Zeeman G.; Pretreatments to enhance the digestibility of lignocellulosic biomass; Bioresource Technology 2009-1:10-8.).

The filamentous fungus that is currently the most widely used as a source of cellulolytic enzyme is the ascomycete Trichoderma reesei. Its secretome (i.e. all of the enzymes secreted by the fungus into the culture medium) mainly contains three types of enzymes, the complementary activity of which allows the hydrolysis of cellulose to glucose: endoglucanases (E.G; EC 3.2.1.4); exoglucanases, especially comprising cellobiohydrolases I and II (CBH; EC 3.2.1.91); β-glucosidases (BGL; EC 3.2.1.21).

For the saccharification of lignocellulose, use is generally made of the entire secretome, in the form of an enzymatic cocktail. The saccharification is performed by simple placing in contact of the lignocellulosic material pretreated with this enzymatic cocktail, and incubation, under optimum temperature and pH conditions for the enzymes concerned for a variable period depending on the nature of the lignocellulosic material concerned and the amount of enzymes used.

The main advantage of Trichoderma reesei lies in its capacity to secrete very large amounts of enzymes. Strains of T. reesei that hypersecrete lignocellulolytic enzymes have been produced by mutagenesis, and their secretome is currently used for the saccharification of lignocellulose. Among these strains, mention will be made especially of the strains MCG77 (U.S. Pat. No. 4,275,167), MCG 80 (ALLEN & ANDREOTTI, Biotechnol Bioeng. 12, 451-459, 1982), RUT C30 (Montenecourt & Eveleigh, Appl. Environ. Microbiol., 34, 777-782, 1977) and CL847 (Warzywoda et al., Biotechnol Bioeng. 25, 3005-3011, 1983).

However, the sequencing and analysis of the genome of T. reesei (Martinez et al., Nat. Biotechnol. 26, 553-60, 2008) have shown that the latter in fact had a certain number of shortcomings, especially in the number and diversity of the genes coding for cellulases, hemicellulases and pectinases, which were smaller than those reported for other filamentous fungi.

It thus appears envisageable to improve the enzymatic cocktail derived from T. reesei by completing it with enzymatic activities that would make it possible to fill in these shortcomings.

It is often considered that one of these shortcomings, in the context of a use for in vitro saccharification, is the low β-glucosidase content of T. reesei. For this reason, it has been proposed to use recombinant strains of T. reesei whose β-glucosidase activity was increased, for example by insertion of several copies of the β-glucosidase gene (PCT WO 92/010 581), by modification of the signal peptide for increasing the amount of β-glucosidase secreted (PCT WO 99/46362) or by mutation of the β-glucosidase gene to produce a more active protein (PCT WO 2010/029 259).

Another approach proposed consists in searching for other cellulolytic fungi, whose secretome might contain enzymatic activities capable of complementing those that appear insufficient in T. reesei, and to make it possible to obtain more efficient saccharification.

In this context, the Inventors have identified a strain of Aspergillus japonicus which satisfies these criteria, and which especially makes it possible, when its secretome is used in combination with that of T. reesei, to significantly increase the production of glucose especially from a pretreated biomass, when compared with the secretome of T. reesei used alone.

This strain, known as CIRM-BRFM 405, was filed under the treaty of Budapest on Jun. 6, 2012, at the CNCM (Collection Nationale de Cultures de Micro-organismes), 25 rue du Docteur Roux, Paris, under the number CNCM I-4639.

One subject of the present invention is, consequently, the use of the strain CNCM I-4639 for obtaining a multi-enzyme preparation containing cellulases and hemicellulases.

More specifically, a subject of the present invention is a multi-enzyme preparation containing cellulases and hemicellulases, characterized in that it contains the secretome of strain CNCM I-4639 of Aspergillus japonicus.

According to a preferred embodiment of the present invention, said secretome may be obtained from a culture of the strain CNCM I-4639 prepared in the presence of a source of carbon inducing the production of lignocellulolytic enzymes, containing arabinoxylans.

Preferred inductive carbon sources are chosen from cereal brans, and/or fractions thereof which may or may not have been autoclaved. Use may be made, for example, of corn, wheat, barley, etc. bran, or a mixture of different cereal brands and/or of fractions thereof. Generally, such an inductive carbon source contains between 14% and 18% by weight of arabinose, between 26% and 30% by weight of xylose, between 0 and 1% by weight of mannose, between 5% and 6% by weight of galactose, between 20% and 24% by weight of glucose, and, where appropriate, between 2% and 4% by weight of ferulic acid.

The other constituents of the culture medium are the usual constituents of media for culturing Aspergillus japonicus, which are known per se to those skilled in the art. Conventionally, these constituents comprise, besides the source of carbon, a source of nitrogen, mineral salts, trace elements, vitamins and, generally, yeast extract.

The secretome of strain CNCM I-4639 may be obtained from a culture of this strain by simple separation of the cells and of the culture supernatant, which contains the secreted proteins. This supernatant may be used in unmodified form, or after simple filtration to free it of the cell debris. However, generally, it will be preferable to concentrate it, for example by diafiltration. The proteins constituting the secretome may also be recovered by precipitation with ammonium sulfate.

The secretome of strain CNCM I-4639 may be used for the saccharification of lignocellulosic biomass, and especially in combination with the secretome of a strain of T. reesei.

Consequently, according to a particularly preferred embodiment of a multi-enzyme preparation in accordance with the invention, it contains the secretome of strain CNCM I-4639 mixed with the secretome of a strain of T. reesei.

Said strain of T. reesei may be, for example, a strain that hypersecretes lignocellulolytic enzymes such as one of the strains MCG77, MCG 80, RUT C30 and CL847 mentioned above. It may also be a recombinant strain such as those described in patent applications PCT WO 92/010 581, PCT WO 99/46362 or PCT WO 2010/029 259.

Methods for producing the secretome of T. reesei are well known per se to those skilled in the art. By way of example, mention will be made of the process described in patent application FR 2 555 603.

The secretome of strain CNCM I-4639 may be mixed with that of a strain of T. reesei in proportions (by weight): proteins of the secretome of CNCM I-4639/proteins of the secretome of T. reesei ranging from 25/75 to 5/95. Advantageously, these proportions will be from 10/90 to 5/95.

A subject of the present invention is also a process for producing fermentable sugars, and especially glucose, from a lignocellulosic substrate, characterized in that it comprises the hydrolysis of said substrate using a multi-enzyme preparation in accordance with the invention, advantageously using a preparation containing the secretome of strain CNCM I-4639 mixed with the secretome of a strain of T. reesei.

The lignocellulosic substrate may be derived from any lignocellulose-rich material, for example farming residues such as cereal straws, lumber residues, materials derived from dedicated cultures such as miscanthus and poplar, residues from the paper industry or from any other industry for transforming cellulosic and lignocellulosic materials. Prior to the hydrolysis, this material is pretreated, as described above, to obtain the lignocellulosic substrate on which the hydrolysis will be performed. The pretreatment is performed in a manner known per se to those skilled in the art, for example according to one of the methods indicated above. A particularly preferred pretreatment method is steam explosion under acidic conditions. The conditions of this pretreatment (amount of acid, pressure and time) are standard conditions, which are known per se to those skilled in the art.

The enzymatic hydrolysis will generally be performed at a temperature of from 30° C. to 50° C., preferably between 37 and 45° C., and at a pH generally between 4.5 and 5.5.

Generally, the reaction mixture contains from 1% to 20% by weight of lignocellulosic substrate dry matter, and the enzymatic preparation in accordance with the invention is used in a proportion of from 5 to 30 mg per gram of substrate (by weight of dry matter).

The duration of the enzymatic hydrolysis may vary especially according to the nature of the substrate and the amount of enzymatic preparation used, and the temperature at which the reaction is performed. It is generally from 24 to 120 hours and preferably from 72 hours to 96 hours. Monitoring of the hydrolysis may be performed by assaying the reducing sugars released and the simple sugars glucose and xylose.

The simple sugars obtained via the process in accordance with the invention may be recovered from the hydrolyzate, for a subsequent use.

Alternatively, the hydrolyzate may be used directly for the production of alcohol, especially of ethanol, by fermentation in the presence of an alcohol-producing microorganism.

A subject of the invention is thus also a process for producing alcohol, especially ethanol, characterized in that it comprises the production, in accordance with the invention, of a hydrolyzate containing fermentable sugars from a lignocellulosic substrate, and the alcoholic fermentation of this hydrolyzate by an alcohol-producing microorganism.

The alcoholic fermentation may be performed, after the enzymatic hydrolysis, under standard conditions that are well known to those skilled in the art.

In general, use is made of an alcohol-producing microorganism such as the yeast Saccharomyces cerevisiae or the bacterium Zymomonas mobilis, and fermentation is performed at a temperature preferably between 30 and 35° C. Alternatively, the alcoholic fermentation may be performed simultaneously with the enzymatic hydrolysis, according to a process of simultaneous saccharification and fermentation known as an SSF process. The operating conditions used in this case for the enzymatic hydrolysis and the alcoholic fermentation differ mainly from those indicated above by the reaction time and temperature. The temperature is generally from 28 to 40° C. and the reaction time is generally from 50 hours to 300 hours.

The invention will be understood more clearly with the aid of the rest of the description that follows, which refers to nonlimiting examples illustrating the properties of the secretome of strain CNCM I-4639.

### EXAMPLE 1

### Search for Microorganisms Capable of Improving the Saccharification Capacities of Trichoderma reesei

The secretomes of various fungal strains of the ascomycetes, basidiomycetes and zygomycetes classes, derived from the collection of the Centre International de Ressources Microbiennes (CIRM-CF; http://www.inra.fr/crb-cirm/), at INRA, Marseille, were tested for their capacity for saccharification of a lignocelluloic substrate, alone or in combination with the secretome of Trichoderma reesei.

The species to which these strains belong, and the number of strains for each species, are listed in Table I below.

TABLE I

| | | Number of strains |
|---|---|---|
| Class | Genus and species | tested |
| Ascomycetes | Aspergillus niger | 5 |
| | Aspergillus japonicus | 2 |
| | Aspergillus wentii | 1 |
| | Aspergillus violaceofuscus | 1 |
| | Penicillium variabilis | 1 |
| | Nectria haematococca | 3 |
| | Haematonectria haematococca | 3 |
| | Trichoderma harzanium | 1 |
| | Chaetomium globosum | 1 |
| Zygomycetes | Rhizopus oryzae | 1 |
| Basidiomycetes | Phellinus sp. | 4 |
| | Gloeoporus pannocinctus | 1 |
| | Ustilago maydis | 1 |
| | Grammothele fuligo | 2 |
| | Polyporus ciliatus | 1 |
| | Trametes sp. | 6 |
| | Trametes gibbosa | 5 |
| | Daedaleopsis confragosa | 5 |
| | Tinctoporellus epimiltinus | 2 |
| | Perenniporia subacida | 1 |
| | Dichomitus squalens | 1 |

The strains are maintained in culture on malt agar in inclined tubes, using the medium MA2 (malt extract at 2% w/v) for the basidiomycetes, and the medium MYA2 (malt extract at 2% w/v and yeast extract at 0.1% w/v) for the ascomycetes and zygomycetes.

### Preparation of the Secretomes:

The strains were cultured in baffled 16-well plates, in liquid medium containing 15 g/l (based on the dry matter) of autoclaved fraction of corn bran (supplied by ARD, Pomacle, France) as source of carbon inducing the production of cellulolytic enzymes, 2.5 g/l of maltose as culture starter, 1.842 g/l of diammonium tartrate as source of nitrogen, 0.5 g/l of yeast extract, 0.2 g/l of KH₂PO₄, 0.0132 g/l of CaCl₂.2H₂O and 0.5 g/l of MgSO₄.7H₂O.

The cultures were inoculated with 2×10⁵ spores/ml for the sporulating fungi, or with mycelium fragments obtained by milling for 40 seconds with a Fastprep®-24 (MP Biomedicals) adjusted to 5 m/s for the non-sporulating fungi. They were then incubated at 30° C. with orbital shaking at 140 rpm (Infors HT, Switzerland) for 7 days for the ascomycetes and 10 days for the basidiomycetes.

The culture medium was harvested, filtered on a polyether sulfone membrane with a pore size of 0.2 μm (Vivaspin®, Sartorius), and then concentrated by diafiltration on a polyether sulfone membrane with a cutoff threshold of 10 kDa (Vivaspin®, Sartorius) in a 50 mM acetate buffer, pH 5, at a final volume of 3 ml and stored at −20° C. until the time of use.

Each diafiltered and concentrated secretome was tested for its capacity to saccharify micronized wheat straw (Triticum aestivum, Apache variety, France). The secretome of strain CL847 of T. reesei (also referred to hereinbelow as enzymatic cocktail E508), supplied by IFPEN (Rueil-Malmaison, France) was used as reference.

The particles of micronized wheat straw have a mean diameter of 100 μm. These particles were suspended at 1% (w/v) in 50 mM acetate buffer, pH 5, supplemented with 40 μg/ml of tetracycline and 30 μg/ml of cycloheximide. The suspension was divided into 96-well plates, which were stored at −20° C. until the time of use.

The saccharification measurements were taken according to the method described by Navarro et al. (Navarro et al., Microbial Cell Factories, 9:58, 2010), using a TECAN GENESIS EVO 200 robot (Tecan).

15 μl of each concentrated secretome (5 to 30 μg of total proteins) were added to the wells of the plate. Each secretome was tested alone, or supplemented with 30 μg of enzymatic cocktail from T. reesei CL847. The reducing sugars released by the saccharification were quantified at the saccharification plateau (24 hours in the case of micronized wheat straw) by assay with DNS. All the reactions were performed independently, at least in triplicate.

The secretomes of 21 of the strains tested, used in combination with that of T. reesei, produced an amount of reducing sugars at least 30% greater than that produced by the secretome of T. reesei alone. These strains, which are listed in Table II below, were selected for the rest of the study.

TABLE II

| | | CIRM-BRFM |
|---|---|---|
| Class | Genus and species | number |
| Ascomycetes | Aspergillus niger | 131 |
| | Aspergillus japonicus | 405 |
| | Aspergillus wentii | 279 |
| | Aspergillus violaceofuscus | 414 |
| | Penicillium variabilis | 110 |
| | Nectria haematococca | 1096 |
| | Haematonectria haematococca | 1286 |
| | Trichoderma harzanium | 866 |
| | Chaetomium globosum | 1103 |
| Zygomycetes | Rhizopus oryzae | 1095 |
| Basidiomycetes | Phellinus sp. | 907 |
| | Gloeoporus pannocinctus | 626 |
| | Ustilago maydis | 1093 |
| | Grammothele fuligo | 1072 |
| | Polyporus ciliatus | 1067 |
| | Trametes sp. | 1120 |
| | Trametes gibbosa | 952 |
| | Daedaleopsis confragosa | 1131 |
| | Tinctoporellus epimiltinus | 1077 |
| | Perenniporia subacida | 750 |
| | Dichomitus squalens | 998 |

### EXAMPLE 2

### Glycosidase Activity Profiles of the Selected Microorganisms

In order to obtain the secretomes in sufficient amount to continue their characterization, the selected strains were cultured in baffled flasks in the inductive medium described above.

100 ml cultures were prepared in 250 ml and 500 ml flasks, respectively, for the ascomycetes and the basidiomycetes. Each culture was inoculated with 2×10⁵ spores/ml for the sporulating fungi, or with 5 ml of mycelium fragments per 100 ml of medium for the non-sporulating fungi. They were then incubated at 30° C. with orbital shaking at 105 or 120 rpm (Infors HT, Switzerland) for 7 days or 10 days for the ascomycetes and the basidiomycetes, respectively.

Each secretome was harvested and filtered as described in Example 1 above. Two successive steps of precipitation with ammonium sulfate at 20% (w/w) and 95% (w/w) were performed. After the second precipitation, the pellet was resuspended in 50 mM acetate buffer, pH 5, concentrated by diafiltration on a polyether sulfone membrane with a cutoff threshold of 10 kDa (Vivaspin, Sartorius) and stored at −20° C. until the time of use.

The proteins were assayed in each secretome, before and after concentration, by Bradford assay (Bio-Rad Protein Assay Dye Reagent Concentrate, Ivry, France) using an SAB calibration range at concentrations from 0.2 to 1 mg/ml.

The protein yields for each of the strains are indicated in Table III below.

TABLE III

| | | Secretome | | | Concentrated secretome | | | | |
|---|---|---|---|---|---|---|---|---|---|
| | | | | Total | | | | Total | Protein |
| | CIRM- | Proteins | Volume | proteins | Proteins | Volume | Concentration | proteins | yield |
| | BRFM | (mg/mL) | (mL) | (mg) | (mg/mL) | (mL) | factor | (mg) | (%) |
| Aspergillus niger | 131 | 0.17 | 1250 | 215 | 9.5 | 22 | 57 | 210 | 98 |
| Penicillium variabilis | 110 | 0.35 | 1295 | 453 | 13.1 | 20 | 65 | 262 | 58 |
| Aspergillus japonicus | 405 | 0.08 | 1290 | 103 | 3.8 | 20 | 65 | 76 | 74 |
| Nectria haematococca | 1096 | 0.29 | 1400 | 406 | 7.6 | 20 | 70 | 152 | 37 |
| Phellinus sp. | 907 | 0.29 | 1300 | 377 | 9.2 | 31 | 42 | 285 | 76 |
| Gloeoporus pannocinctus | 626 | 0.22 | 1350 | 297 | 14.3 | 20 | 68 | 285 | 96 |
| Trichoderma harzanium | 866 | 0.18 | 1330 | 234 | 6.9 | 31 | 43 | 215 | 92 |
| Ustilago maydis | 1093 | 0.18 | 1370 | 247 | 6.3 | 36 | 38 | 229 | 93 |
| Rhizopus oryzae | 1095 | nd | 280 | nd | 2.3 | 27.5 | 10 | 63 | nd |
| Aspergillus wentii | 279 | 0.09 | 1215 | 111 | 3.5 | 21.5 | 57 | 74 | 67 |
| Aspergillus violaceofuscus | 414 | 0.16 | 1310 | 212 | 5.4 | 27.5 | 48 | 148 | 70 |
| Grammothele fuligo | 1072 | nd | 1165 | nd | 4.7 | 30 | 39 | 140 | nd |
| Haematonectria haematococca | 1286 | 0.23 | 1390 | 318 | 5.1 | 32 | 43 | 163 | 51 |
| Chaetomium globosum | 1103 | 0.16 | 1315 | 210 | 6.1 | 36 | 37 | 219 | 100 |
| Polyporus ciliatus | 1067 | 0.20 | 1300 | 260 | 6.7 | 22 | 59 | 147 | 56 |
| Trametes sp. | 1120 | 0.23 | 1200 | 276 | 5.5 | 20 | 60 | 110 | 40 |
| Daedaleopsis confragosa | 1131 | 0.21 | 1100 | 230 | 1.7 | 76 | 14 | 126 | 55 |
| Tinctoporellus epimiltinus | 1077 | 0.19 | 1320 | 244 | 1.7 | 66 | 20 | 115 | 47 |
| Perenniporia subacida | 750 | 0.25 | 1335 | 330 | 3.0 | 100 | 13 | 303 | 92 |
| Dichomitus squalens | 998 | 0.25 | 1340 | 335 | 6.2 | 39 | 34 | 242 | 72 |
| Trametes gibbosa | 952 | 0.11 | 1235 | 135 | 2.4 | 50 | 25 | 122 | 91 |

The concentrated secretomes were tested for their glycoside hydrolase activities on various substrates. The cellulose degradation was estimated by quantifying the endo-glucanase activities (carboxymethyl cellulose, CMC), Avicelase (Avicel, AVI), FPase (filter paper, FP), cellobiohydrolase (pNP-β-D-cellobioside, pCel and pNP-β-D-lactobioside, pLac) and β-glucosidase (pNP-β-D-glucopyranoside, pGlc). The hemicellulose degradation was evaluated by quantification of the xylanases and mannanases using various xylans and mannans as substrates. The main exoglycosidase activities were evaluated by quantifying the hydrolysis of pNP-α-L-arabinofuranoside (pAra), pNP-α-D-galactopyranoside (pGal), pNP-β-D-xylopyranoside (pXyl) and pNP-β-D-mannopyranoside (pMan). The pectin degradation was determined using arabinogalactan and arabinan as substrates, and the global esterase activity was determined on pNP-acetate (pAc).

For pNPs pGlc, pLac, pCel, pXyl, pAra, pGal, and pMan (Sigma), a 1 mM solution of pNP in 50 mM acetate buffer, pH 5, was distributed in the wells of a 96-well polystyrene plate, in an amount of 100 μl per well, and one column per substrate. A range of 0 to 0.2 mM of pNP used as calibration was added to each plate. The plates were frozen at −20° C. until the time of use.

Assay was performed by adding 20 μl of each secretome to the pNP plates, preincubated at 37° C. The plates were then sealed using a PlateLoc device (Velocity 11, Agilent) to prevent evaporation, and incubated at 37° C. with shaking at 1000 rpm (Mixmate, Eppendorf). After 30 minutes, the reaction was stopped by addition of 130 μl of a 1M Na₂CO₃ solution, pH 11.5. The amount of pNP released was measured at 410 nm and quantified relative to the pNP calibration range. In the case of pAc (Sigma), a storage solution at 20 mM in DMSO was diluted to 1 mM in 50 mM sodium phosphate, pH 6.5, immediately before use. 15 μl of each secretome were added, and the hydrolysis kinetics were monitored by measuring the absorbance at 410 nm over one minute.

One enzyme unit was defined as 1 μmol of p-nitrophenyl released per mg of protein and per minute under the experimental conditions used.

The complex substrates used are carboxymethylcellulose (CMC, Sigma), Avicel PH101 (Fluka), birch xylan (BirchX, Sigma), low-viscosity wheat xylan (WheatX, Megazyme, Wicklow, Ireland), insoluble wheat arabinoxylan (WheatXI, Megazyme), insoluble ivory palm kernel seed mannan (MAN, Megazyme), locust beam galactomannan (GalMan, Megazyme), larch arabinogalactan (AraGal, Megazyme) and sugar beet arabinan (Megazyme).

A solution or suspension at 1% w/v of each of these substrates in 50 mM acetate buffer, pH 5, was distributed in the wells of a 96-well polystyrene plate, at an amount of 100 μl per well, and one column per substrate. A range from 0 to 20 mM of glucose used as calibration was added to each well. The plates were frozen at −20° C. until the time of use. Assay was performed by adding 20 μl of each secretome to the plates preincubated at 37° C. The plates were then incubated at 37° C. with shaking in the Tecan Genesis Evo 200 robotic incubator (Tecan France, Lyons, France) for 1 hour. The reducing sugars were quantified by assay with DNS, using the automated method described by Navarro et al. (2010, mentioned above). One enzyme unit was defined as 1 μmol of glucose equivalent released per mg of protein and per minute under the experimental conditions used.

The global cellulase activity was determined on filter paper disks (Whatmann No. 1) 6 mm in diameter. Flasks each containing a filter paper disk in 100 μl of 50 mM acetate buffer, pH 5, and 50 μl of secretome tested were incubated for 2 hours at 50° C. All the tests were performed in triplicate. After incubation, the reducing sugars were quantified by assay with DNS as described above. One enzyme unit was defined as 1 μmol of glucose equivalent released per mg of protein and per minute under the experimental conditions used.

The results for all of the strains tested are summarized in Table IV below.

TABLE IV

| | E508 | 131 | 110 | 405 | 1096 | 907 | 626 | 866 | 1093 | 1095 | 279 |
|---|---|---|---|---|---|---|---|---|---|---|---|
| | T. ree | A. nig | P. var | A. jap | N. hae | P. sp. | G. pan | T. har | U. may | R. ory | A. wen |
| CMC | 0.33 | 0.35 | 0.09 | 0.69 | 0.07 | 0.14 | 0.08 | 0.08 | 0.03 | 0.01 | 0.14 |
| Avicel | 0.01 | 0.06 | 0.01 | 0.01 | 0.01 | 0.00 | 0.01 | 0.00 | 0.00 | 0.02 | 0.01 |
| Filter | 0.12 | 0.15 | 0.02 | 0.16 | 0.05 | 0.05 | 0.04 | 0.06 | 0.08 | 0.06 | 0.16 |
| Pectin | 0.12 | 2.36 | 0.19 | 0.23 | 0.17 | 0.19 | 0.18 | 0.27 | 0.38 | 0.78 | 0.26 |
| paper | | | | | | | | | | | |
| BirchX | 0.94 | 41.90 | 2.14 | 4.10 | 0.25 | 0.77 | 0.25 | 0.87 | 3.93 | 0.09 | 9.09 |
| WheatX | 1.59 | 103.60 | 3.86 | 7.25 | 0.53 | 1.38 | 0.33 | 1.67 | 3.75 | 0.20 | 13.04 |
| WheatXI | 0.37 | 22.49 | 0.97 | 1.93 | 0.09 | 0.15 | 0.07 | 0.50 | 1.29 | 0.01 | 4.27 |
| Man | 0.01 | 0.25 | 0.07 | 0.01 | 0.00 | 0.18 | 0.53 | 0.01 | 0.00 | 0.05 | 0.02 |
| GalMan | 0.02 | 0.75 | 0.22 | 0.02 | 0.01 | 0.45 | 0.95 | 0.04 | 0.00 | 0.16 | 0.03 |
| Arabinan | 0.01 | 0.41 | 0.28 | 0.09 | 0.07 | 0.07 | 0.04 | 0.08 | 0.02 | 0.03 | 0.06 |
| AraGal | 0.01 | 0.10 | 0.08 | 0.20 | 0.12 | 0.16 | 0.13 | 0.11 | 0.18 | 0.16 | 0.08 |
| pNP-Glc | 0.19 | 0.58 | 3.73 | 1.95 | 0.03 | 1.43 | 0.03 | 0.18 | 0.01 | 0.01 | 0.20 |
| pNP-Lac | 0.04 | 0.02 | 0.20 | 0.21 | 0.01 | 0.04 | 0.01 | 0.05 | 0.01 | 0.01 | 0.00 |
| pNP-Cel | 0.05 | 0.15 | 0.14 | 0.25 | 0.01 | 0.09 | 0.01 | 0.01 | 0.01 | 0.02 | 0.01 |
| pNP-Xyl | 0.01 | 0.07 | 0.51 | 0.08 | 0.00 | 0.03 | 0.01 | 0.02 | 0.09 | 0.01 | 0.01 |
| pNP-Ara | 0.02 | 0.42 | 0.68 | 0.05 | 0.00 | 0.05 | 0.03 | 0.01 | 0.24 | 0.01 | 0.01 |
| pNP-Gal | 0.01 | 0.53 | 38.8 | 0.50 | 0.00 | 0.46 | 0.04 | 0.19 | 0.93 | 0.01 | 0.13 |
| pNP-Man | 0.00 | 0.02 | 0.04 | 0.01 | 0.00 | 0.02 | 0.01 | 0.01 | 0.01 | 0.01 | 0.01 |
| pNP-Ac | 0.00 | 2.09 | 1.31 | 0.65 | 0.19 | 0.27 | 0.00 | 0.08 | 0.01 | 0.06 | 0.42 |

| | 414 | 1072 | 1286 | 1103 | 1067 | 1120 | 1131 | 1077 | 750 | 998 | 952 |
|---|---|---|---|---|---|---|---|---|---|---|---|
| | A. vio | G. ful | H. hae | C. glo | P. cil | T. sp. | D. con | T. epi | P. sub | D. squ | T. gib |
| CMC | 0.08 | 0.03 | 0.07 | 0.03 | 0.04 | 0.01 | 0.01 | 0.06 | 0.16 | 0.21 | 0.04 |
| Avicel | 0.02 | 0.01 | 0.01 | 0.01 | 0.01 | 0.01 | 0.00 | 0.03 | 0.03 | 0.02 | 0.08 |
| Filter paper | 0.06 | 0.08 | 0.07 | 0.04 | 0.07 | 0.03 | 0.11 | 0.06 | 0.07 | 0.08 | 0.24 |
| Pectin | 0.17 | 0.69 | 0.17 | 0.14 | 0.24 | 0.12 | 0.76 | 0.74 | 0.38 | 0.25 | 0.28 |
| BirchX | 0.08 | 0.33 | 0.11 | 0.04 | 0.18 | 0.06 | 0.60 | 0.35 | 0.87 | 0.53 | 0.18 |
| WheatX | 0.12 | 0.71 | 0.10 | 0.06 | 0.39 | 0.10 | 1.06 | 0.92 | 1.35 | 0.93 | 0.29 |
| WheatXI | 0.02 | 0.10 | 0.02 | 0.01 | 0.04 | 0.02 | 0.09 | 0.11 | 0.21 | 0.13 | 0.10 |
| Man | 0.02 | 0.03 | 0.01 | 0.00 | 0.05 | 0.01 | 0.02 | 0.04 | 1.04 | 0.53 | 0.06 |
| GalMan | 0.04 | 0.08 | 0.02 | 0.01 | 0.19 | 0.04 | 0.04 | 0.08 | 1.72 | 0.96 | 0.10 |
| Arabinan | 0.01 | 0.27 | 0.07 | 0.01 | 0.17 | 0.09 | 0.14 | 1.26 | 0.42 | 0.17 | 0.08 |
| AraGal | 0.27 | 0.39 | 0.21 | 0.18 | 0.12 | 0.15 | 0.24 | 0.15 | 0.17 | 0.16 | 0.47 |
| pNP-Glc | 0.13 | 0.02 | 0.07 | 0.10 | 0.05 | 0.05 | 0.05 | 0.25 | 0.07 | 0.14 | 0.03 |
| pNP-Lac | 0.00 | 0.01 | 0.00 | 0.00 | 0.00 | 0.01 | 0.00 | 0.06 | 0.01 | 0.02 | 0.00 |
| pNP-Cel | 0.00 | 0.01 | 0.01 | 0.00 | 0.00 | 0.00 | 0.22 | 0.01 | 0.02 | 0.01 | 0.00 |
| pNP-Xyl | 0.00 | 0.01 | 0.00 | 0.00 | 0.01 | 0.01 | 0.04 | 0.02 | 0.02 | 0.00 | 0.01 |
| pNP-Ara | 0.00 | 0.65 | 0.02 | 0.00 | 0.17 | 0.01 | 0.01 | 1.18 | 0.28 | 0.03 | 0.02 |
| pNP-Gal | 0.00 | 1.58 | 0.00 | 0.00 | 0.49 | 0.12 | 0.09 | 1.05 | 1.32 | 0.46 | 0.00 |
| pNP-Man | 0.00 | 0.01 | 0.00 | 0.00 | 0.02 | 0.01 | 0.00 | 0.08 | 0.01 | 0.09 | 0.00 |
| pNP-Ac | 0.02 | 0.18 | 0.17 | 0.00 | 0.34 | 0.00 | 0.00 | 0.36 | 0.41 | 0.43 | 0.00 |

### EXAMPLE 3

### Capacity of the Secretomes of the Selected Microorganisms to Complement the Secretome of Trichoderma reesei for the Production of Glucose and Xylose

The secretomes of the 24 strains selected were tested for their capacity to release glucose and xylose from a lignocellulosic substrate, alone or in combination with the secretome of Trichoderma reesei (enzymatic cocktail E508 of the strain CL847).

The saccharification tests and the assay of the reducing sugars released were performed on micronized wheat straw, as described in Example 1 above. The glucose and xylose were quantified by high-performance anion-exchange chromatography on a CarboPac PA-1 column (Dionex, Voisins-le-Bretonneux, France).

The results are collated in Table V below. They are expressed as a percentage of those obtained with the enzymatic cocktail E508, used as reference.

TABLE V

| | | | | | | | | | | |
|---|---|---|---|---|---|---|---|---|---|---|
| E508 | 131 | 110 | 405 | 1096 | 907 | 626 | 866 | 1093 | 1095 | 279 |
| T. ree | A. nig | P. var | A. jap | N. hae | P. sp. | G. pan | T. har | U. may | R. ory | A. wen |

Secretome alone

| | | | | | | | | | | | |
|---|---|---|---|---|---|---|---|---|---|---|---|
| Total sugars | 100 | 115 | 65 | 92 | 95 | 64 | 111 | 73 | 131 | 56 | 80 |
| Glucose | 100 | 84 | 34 | 45 | 17 | 28 | 27 | 21 | 51 | 28 | 31 |
| Xylose | 100 | 80 | 42 | 84 | 35 | 27 | 49 | 43 | 79 | 60 | 99 |

As a mixture with the secretome from Trichoderma reesei

| | | | | | | | | | | | |
|---|---|---|---|---|---|---|---|---|---|---|---|
| Total sugars | 100 | 142 | 135 | 158 | 148 | 175 | 192 | 158 | 195 | 144 | 155 |
| Glucose | 100 | 104 | 91 | 110 | 85 | 85 | 82 | 86 | 118 | 92 | 108 |
| Xylose | 100 | 93 | 77 | 101 | 82 | 84 | 106 | 80 | 123 | 109 | 104 |

| | 414 | 1072 | 1286 | 1103 | 1067 | 1120 | 1131 | 1077 | 750 | 998 | 952 |
|---|---|---|---|---|---|---|---|---|---|---|---|
| | A. vio | G. ful | H. hae | C. glo | P. cil | T. sp. | D. con | T. epi | P. sub | D. squ | T. gib |
| Secretome alone | | | | | | | | | | | |
| Total sugars | 89 | 96 | 84 | 60 | 52 | 52 | 28 | 44 | 37 | 45 | 37 |
| Glucose | 41 | 23 | 28 | 27 | 31 | 26 | 23 | 26 | 26 | 29 | 16 |
| Xylose | 22 | 25 | 69 | 26 | 37 | 21 | 18 | 25 | 23 | 25 | 36 |
| As a mixture with the secretome from Trichoderma reesei | | | | | | | | | | | |
| Total sugars | 169 | 174 | 206 | 102 | 112 | 111 | 108 | 124 | 110 | 106 | 108 |
| Glucose | 85 | 98 | 109 | 109 | 88 | 96 | 104 | 109 | 99 | 78 | 108 |
| Xylose | 70 | 94 | 164 | 123 | 76 | 108 | 89 | 95 | 96 | 59 | 149 |

These results show especially that the secretomes of the strains of Aspergillus nidulans, Aspergillus wentii and Aspergillus japonicus CIRM-BRFM 405 (CNCM I-4639) are among the best for complementing the secretome of T. reesei in order to release large amounts of glucose.

### EXAMPLE 4

### Complementation of a Secretome of T. reesei with Secretomes of Several Fungal Strains for the Release of Glucose from Pretreated Wheat Straw

Several secretomes show an effect of complementation of the secretome of Trichoderma reesei for the hydrolysis of native straw. Among these, several were prepared as described in Example 2 above and were tested for their capacities for complementing the secretome of T. reesei for the release of glucose from a substrate of industrial type (pretreated wheat straw). The secretomes studied in example 4 are those produced by the strains of Aspergillus nidulans, Aspergillus wentii and Aspergillus japonicus CIRM-BRFM 405.

Pretreatment of the wheat straw was performed by steam explosion under acidic conditions. The raw straw was soaked in 0.04 M H₂SO₄ solution for 16 hours and then subjected to a steam explosion treatment in a batchwise autohydrolysis reactor, for 150 s at 20 bar and 210° C. After 2 washes with water, the straw was subjected to a pressure of 100 bar for 3 minutes to obtain a dry matter content of about 30%.

The T. reesei cocktail used in this example is batch K616 produced by the strain T. reesei CL847 iβ. It has the feature of having a better level of β-glucosidase activity than batch E508 produced by T. reesei CL847, since the strain T. reesei iβ integrates a vector that overexpresses native β-glucosidase (specific activities of K616: FPU (filter paper unit) activity: 0.67 IU/mg; PNPGU (para-nitrophenyl-β-D-glucose hydrolysis) activity: 4.6 IU/mg).

The hydrolysis tests were performed in 10 ml glass flasks. 250 mg of screened and lyophilized substrate were suspended in a total volume of 5 ml containing 50 mM of pH 4.8 citrate buffer (Merck, Prolabo) and 50 μl of chloramphenicol (30 g l-1) (Sigma-Aldrich). The flasks were incubated at 45° C. for 30 minutes before addition of the secretomes. The secretome of T. reesei was used at a concentration of 10 mg of protein per gram of substrate. Supplementation with the secretome of the strains of Aspergilli was performed at a rate of 7% by weight of the added T. reesei proteins. The flasks were reincubated at 45° C. with shaking at 175 rpm and samples were taken between 0 and 72 hours. After inactivation of the enzymes in boiling water for 5 minutes, and centrifugation, the supernatants were filtered and the glucose production measured by high-performance anion-exchange chromatography on a CarboPac PA-1 column (Dionex).

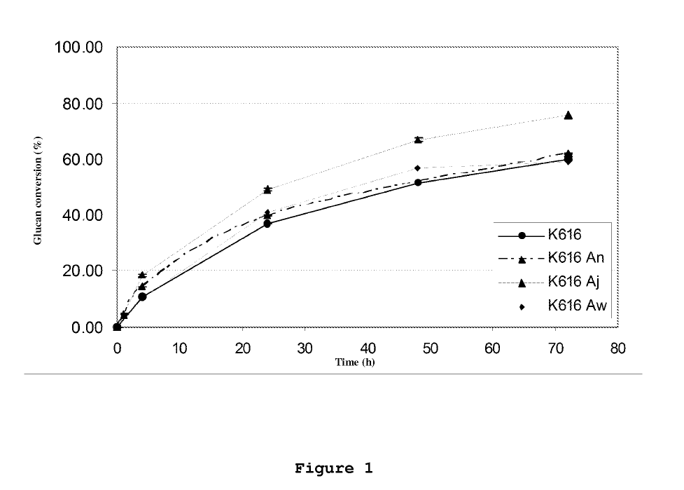

The results are illustrated in FIG. 1. These results show that only the secretome of the strain of A. japonicus CIRM-BRFM 405 makes it possible to improve the glucose release capacities.

### EXAMPLE 5

### Complementation of the Secretomes of T. reesei with the Strain of A. japonicus CIRM-BRFM 405 (CNCM I-4639) for the Release of Glucose from Pretreated Wheat Straw

In order to determine whether the properties of the secretome of the strain CIRM-BRFM 405 could be attributed to an effect of supplementation with β-glucosidase activity of the secretome of T. reesei, the secretomes of two strains of T. reesei were used. The first secretome is cocktail K616 used in example 4. The second secretome, referred to as enzymatic cocktail K667, was produced by a transformed strain of T. reesei containing an improved β-glucosidase with strong activity, as described in patent application PCT WO 2010/029 259 (specific activities of K667: FPU 0.68 IU/mg; PNPGU 12.5 IU/mg).

The hydrolysis tests were performed as described in example 4. The secretome of T. reesei was used at a concentration of 10 mg of protein per gram of substrate. Supplementation with the secretome from the strain of A. japonicus was performed at an amount of 7% by weight of added T. reesei proteins. The flasks were reincubated at 45° C. with shaking at 175 rpm and samples were taken at 0, 4, 24, 48 and 72 hours. After inactivation of the enzymes with boiling water for 5 minutes, and centrifugation, the supernatants were filtered and the glucose production measured by high-performance anion-exchange chromatography on a CarboPac PA-1 column (Dionex).

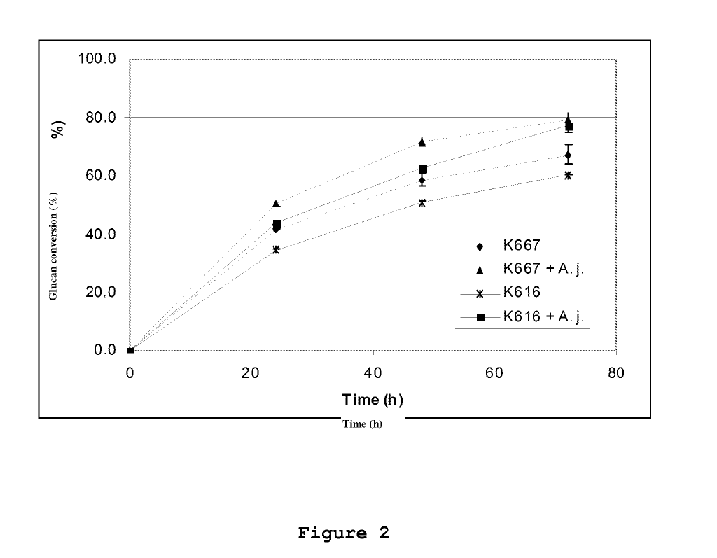

The results are illustrated in FIG. 2. These results show that, irrespective of the T. reesei secretome used, the secretome from the strain of A. japonicus CIRM-BRFM 405 makes it possible to improve the glucose release capacities, and that this improvement appears to be independent of an effect of supplementation with β-glucosidase activity.

1. A multi-enzyme preparation containing cellulases and hemicellulases, characterized in that it contains the secretome of the strain CNCM I-4639 from Aspergillus japonicus.

2. The preparation as claimed in claim 1, characterized in that said secretome is obtained from a culture of the strain CNCM I-4639 prepared in the presence of a source of carbon that induces the production of lignocellulolytic enzymes, wherein the source of carbon contains arabinoxylans.

3. The preparation as claimed in claim 2, characterized in that said inductive source of carbon is chosen from cereal brans, fractions of cereal brans, or mixtures thereof.

4. The multi-enzyme preparation as claimed in claim 1, characterized in that it also contains the secretome of a strain of Trichoderma reesei.

5. A method for saccharification of a lignocellulosic substrate, characterized in that the substrate is contacted with a multi-enzyme preparation as claimed in claim 1.

6. A process for producing fermentable sugars from a lignocellulosic substrate, characterized in that the process comprises hydrolysis of said substrate using a multi-enzyme preparation as claimed in claim 1.

7. A process for producing alcohol from a lignocellulosic substrate, characterized in that the process comprises production of a hydrolyzate containing fermentable sugars via a process as claimed in claim 6, and the alcoholic fermentation of said hydrolyzate by an alcohol-producing microorganism.

8. The multi-enzyme preparation as claimed in claim 2, characterized in that it also contains the secretome of a strain of Trichoderma reesei.

9. The multi-enzyme preparation as claimed in claim 3, characterized in that it also contains the secretome of a strain of Trichoderma reesei.

10. A method for saccharification of a lignocellulosic substrate, characterized in that the substrate is contacted with a multi-enzyme preparation as claimed in claim 2.

11. A method for saccharification of a lignocellulosic substrate, characterized in that the substrate is contacted with a multi-enzyme preparation as claimed in claim 3.

12. A method for saccharification of a lignocellulosic substrate, characterized in that the substrate is contacted with a multi-enzyme preparation as claimed in claim 4.

13. A process for producing fermentable sugars from a lignocellulosic substrate, characterized in that the process comprises hydrolysis of said substrate using a multi-enzyme preparation as claimed in claim 2.

14. A process for producing fermentable sugars from a lignocellulosic substrate, characterized in that the process comprises hydrolysis of said substrate using a multi-enzyme preparation as claimed in claim 3.

15. A process for producing fermentable sugars from a lignocellulosic substrate, characterized in that the process comprises hydrolysis of said substrate using a multi-enzyme preparation as claimed in claim 4.

16. The process for producing alcohol of claim 7 from a lignocellulosic substrate, characterized in that hydrolysis of said substrate uses a preparation containing a secretome obtained from a culture of the strain CNCM I-4639 prepared in the presence of a source of carbon that induces the production of lignocellulolytic enzymes, wherein the source of carbon containing contains arabinoxylans.

17. The process as claimed in claim 16, characterized in that said inductive source of carbon is chosen from cereal brans, and fractions of cereal brans, or mixtures thereof.

18. The process as claimed in claim 16, characterized in that the preparation also contains the secretome of a strain of Trichoderma reesei.
